(19) United States
(12) Patent Application Publication
(10) Pub. No.: US 2020/0143497 A1
(43) Pub. Date: May 7, 2020

(54) Reservation method utilizing a software platform for a consumer-user to instantly book and pay for a birth or pregnancy related classes or sessions or services with a provider listed within the software platform

(71) Applicant: Jessica Lynn Greenhalgh, San Gabriel, CA (US)

(72) Inventor: Jessica Lynn Greenhalgh, San Gabriel, CA (US)

(21) Appl. No.: 16/669,520

(22) Filed: Oct. 31, 2019

Related U.S. Application Data
(60) Provisional application No. 62/756,593, filed on Nov. 7, 2018.

(51) Int. Cl. G06Q 50/20; G06Q 10/02; G06Q 30/06; G16H 10/60

(52) U.S. Cl. CPC G06Q 50/20; G06Q 10/02; G06Q 30/0631; G06Q 30/0619; G06Q 20/3221; G06Q 30/0639; G06Q 30/0623; G16H 10/60; G06Q 30/0605

(57) ABSTRACT

A reservation method utilizing a software platform which enables a consumer-user to instantly schedule, book, and pay for birth and pregnancy related classes, sessions, and services fulfilled by birth and pregnancy providers listed within the software platform system. The customer booked service is assigned to a provider listed within the software platform system, the provider is immediately dispatched and notified of the consumer booked service information, and the provider fulfills the booked service for the customer as scheduled. The method does not require the consumer to make prior contact with the birth or pregnancy provider prior to instantly booking and scheduling the service, and the software platform is user accessible on various devices including a computer or mobile device. The provider also receives important consumer related information with each consumer booked service, including pregnancy due date information entered by the consumer within the software platform for the provider to receive.

### CROSS REFERENCE TO RELATED APPLICATIONS

This application claims the benefit of U.S. Provisional Application No. 62/756,593, titled “Reservation method utilizing a software platform to allow for a consumer-user to instantly book and pay for a birth or pregnancy related classes or sessions or services with a provider listed within the software platform.”, by inventor Jessica L Lynn Greenhalgh, filed on Nov. 7, 2018, the disclosures of which are incorporated herein by reference in their entireties.

### FIELD OF THE INVENTION

The method of the present invention relates to a reservation method utilizing a software platform for consumer-users to instantly book and pay for birth and pregnancy related classes, sessions, and services with various birth and pregnancy providers listed in the software platform system.

### BACKGROUND OF THE INVENTION

The method of using software to book a service has been previously used in certain industries for specific services, including hotel booking, food ordering, and on demand transportation service booking. However, using an automated reservation method utilizing a software platform system for a consumer to instantly book a variety of birthing and pregnancy related services, sessions, and classes from a list of multiple birth-pregnancy providers listed on a software platform system without previously finding or making contact with a provider had never previously been invented, used, designed, nor available, other than with the present invention. The present invention offers an entirely new method to the birthing and pregnancy industry by giving consumers the ability to book and purchase birth and pregnancy related services instantly on-demand. Prior to the present invention, the process for a consumer to find and book a service with a birthing or pregnancy provider involved the consumer having to manually find each provider through a phonebook, personal referral, an internet search or website, and then individually make some form of contact with the provider directly, either through their website, by email, or telephone to inquiry about the provider's specific services available, to book services with the provider, or to determine the provider's available schedule. This manual method is time consuming and strenuous as it requires the customer to individually find and contact each provider, determine the exact type of services the provider offers, and then inquire into receiving scheduling services based on the provider's own available schedule. Instead, the present invention makes it very easy for the consumer to use the software platform system to select the desired service type, pay and book with one of multiple providers listed in the system. This present invention method further makes it easier for a consumer to book a service based on their own schedule rather than the schedule of the provider, and the method of the present invention does not require the consumer to make prior contact with the birth-pregnancy provider prior to instantly booking and scheduling the service. The type of services a consumer may book using the present invention include midwife services, doula services, lactation consultant services, at home birth services, private birth center services, lactation instruction sessions, group and private breastfeeding classes, at home lactation consulting, at home insemination services, at home doula services, pregnancy classes, pregnancy fitness classes, birthing classes, at home water birth services, prenatal and postnatal care classes, and pregnancy nutrition sessions.

There has also been a lack of modern device accessibility usage for consumers looking to book birth and pregnancy related services, as there was no prior method available nor invented that allowed for the customer to instantly schedule and book a birth or pregnancy service on the consumer's computer or mobile device without the consumer first manually finding or make prior contact with a specific provider. The present invention is not only useful for giving consumers the method and ability to instantly schedule, book and pay for various birth and pregnancy related services with multiple providers listed within the software platform without needing to find and or make prior contact with a provider, but it also increases device accessibility when booking birth and pregnancy related services since the software platform application can be accessed by the consumer either through a computer connected to the internet that is logged onto the website where the software platform application is installed, or else through a mobile device having the reservation software platform application installed.

### SUMMARY OF THE INVENTION

An embodiment of the present invention provides a reservation method using a software platform system to allow for consumers to instantly schedule, book and pay for birth or pregnancy related classes, sessions and services with providers listed in the software platform's reservation system. The consumer does not need to make prior contact with the provider before booking the service in the software platform, which provides on-demand instant booking of birth and pregnancy related services. The reservation software platform may be accessed on the internet through a website containing the software platform, or through a mobile device having the software platform application installed. Depending on the class, session or service booked by the consumer, the provider will either go to the customer's location to provide the class, session or service, or the customer will go to the provider's location at the scheduled time. The provider will pay a referral fee to the operator of the software platform system for receiving the assignment of each booked service, session or service that was booked by the customer using the software platform's reservation method. The providers are able to sign-up and be added into the software platform system. The consumer booked services are immediately scheduled and sent to the birthing-pregnancy provider upon the customer booking the available class, session or service available in the reservation software platform system. The software platform system is accessible to both the consumer and provider. Within the software platform, the providers are able to individually list their available time and day schedules for receiving bookings of the specific classes, sessions, and/or services which they provide. The customer is able to use the reservation method to find, reserve/book, and pay for an available class, session, or service with a provider listed within the software platform system. The consumer is able to book and make payment for the booked service within the software platform system. Upon the consumer making a booking, the provider matched to the booking is immediately dispatched and sent an automated message informing the provider of the new booking along with receiving the consumer's booking information. The software platform system will also notate the provider no longer has a time/date block available at the time booked by the customer so that double bookings will not accidentally occur within the system. The birth and pregnancy related classes, sessions and service available for booking in the system includes midwife services, doula services, at home birth services, birth center services, at home insemination services, lactation consulting services, birth coaching, breastfeeding, childbirth classes, infant parenting classes, pregnancy exercise classes and sessions, pregnancy nutrition sessions, scheduled births to take place at birthing centers, scheduled home birth sessions, specialty birth sessions including water births.

### BRIEF DESCRIPTION OF THE DRAWINGS

FIGS. 1A-1B is a block diagram illustrating the embodiment of the present invention.

### DETAILED DESCRIPTION OF THE INVENTION

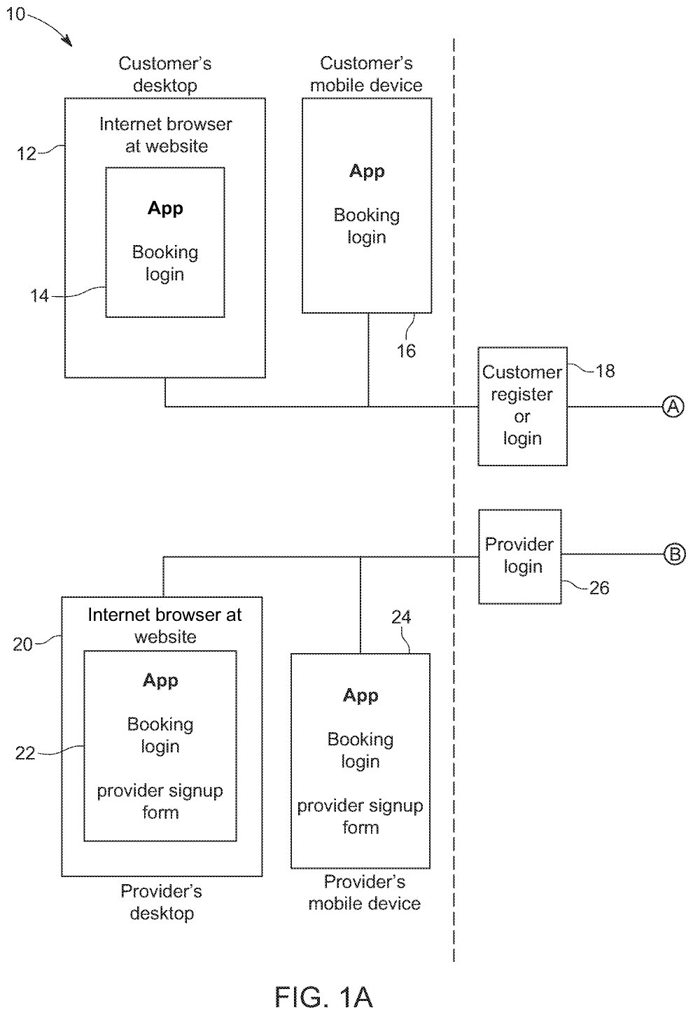

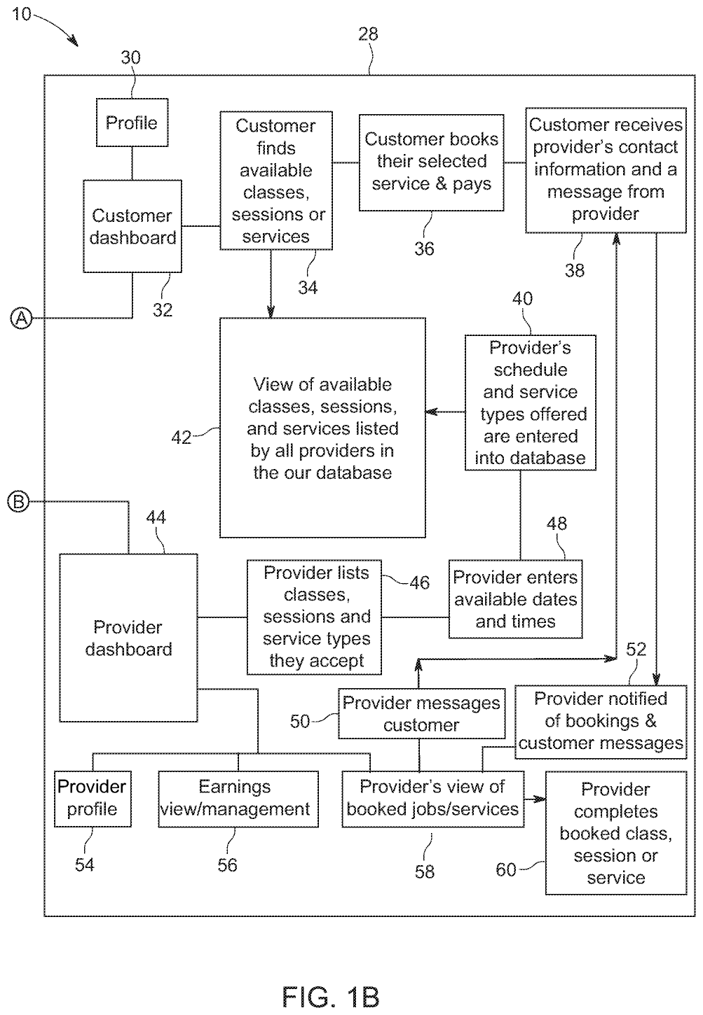

The present invention provides a reservation method and system for instantly booking birth and pregnancy related services using a software platform system having a client/server environment with a user-friendly graphical user interface. This platform software system provides a consumer the ability to instantly book a variety and birth and pregnancy related services from a selection of multiple providers from various locations available within the software platform system, without requiring the consumer to contact the provider prior to booking the service. As used herein, the term “provider” is used in a broad sense and is meant to include midwives, doulas, home birth midwives, nurse-midwives, lactation consultants and counselors, birthing coaches, pregnancy fitness coaches, pregnancy nutrition specialists, prenatal and post natal specialists, insemination specialists, or any other provider that is a birthing or pregnancy specialist. A provider may or may not have an at home service associated with it, where the at home service involves the provider fulfilling the services at the consumers own specified location, including the home of the consumer. Further, a provider may or may not have their own private facility where the service is fulfilled for the customer. Further as used herein, the term “service” used in a broad sense and is meant to include a class, session, procedure or service in either a private or group setting taking place at either the consumer location or provider location, depending on the specific service booked. Although the invention has been described in terms of a specific embodiment, it will be readily apparent to those skilled in this art that various modifications, rearrangements and substitutions can be made without departing from the spirit of the invention. The scope of the invention is defined by the claims appended hereto. FIGS. 1A and 1B are block diagrams that together illustrate the reservation method utilizing a software platform for instantly booking birth and pregnancy related services, classes or sessions with a birth-pregnancy provider. Referring now to FIG. 1A, it exhibits using the reservation method for booking a birth or pregnancy service with a consumer-user accessing the software platform through a computer device 12 having internet access and connected to the website containing the software platform application, or through a mobile device 16 having the software platform application installed on the device. The software platform application provides a login screen feature 18 for the consumer-user to log in to the consumer side of the software platform system 10. The consumer-user can create an account or logs in with a previously made account at the login screen 18. The provider is given access to different features within the software platform application by having a provider login 26, with the provider accessing the software platform through a computer with an internet browser connected to the website 20 where the software platform is installed, or through a mobile device having the software platform application installed 24. After the consumer-user or provider-user access the software platform application as in FIG. 1A, the steps for booking, paying, and fulfillment of a booked service, class or session using the graphical user interface of the software platform can be best understood with reference to FIG. 1B. Referring now to FIG. 1B., the consumer-user accesses the customer dashboard 32 after having logged into the graphical user interface dashboard 28 of the software platform 10. From the customer dashboard 32 the consumer can access their profile settings 30 or they can access the find viewer 34 to search for specific birth and pregnancy related classes, sessions or services available to book, along with searching for specific providers based on location and service type. The consumer can also access the master list view 42 that shows all available classes, sessions and services by all providers in the system database. Upon the customer finding a service to book, the customer is able to select the service and then be sent to the booking checkout feature 36 to immediately book and pay for the selected birth or pregnancy related service. After the service is booked, the software platform's booking messaging feature 38 automatically and immediately providers the consumer with the contact information belonging to the provider scheduled to fulfill the consumer's booked service. The provider is immediately notified of the consumer's booked service via the provider notification feature 52, and the provider notification feature 52 will also immediately notify the provider of any messages sent to the provider by the consumer using the messaging feature. The booked service is immediately and automatically entered and displayed on the provider's booking view management feature 58. The provider messages the customer 50 to discuss detailed information regarding the consumer's booked service and to connect with the consumer prior to fulfilling the booked service. The provider is able to use the earning management view feature 56 to view total earnings earned from fulfilling each booking along with viewing payments received minus the referral fee charged by the operator of the reservation platform system for each service booked by a consumer. The provider profile view 54 is used for the provider to view and upload their profile information to be displayed to the consumers. The provider-user uses the service type list 46 to enter the birth and pregnancy service, class, and session types they offer and accept. After the provider-user entering the type of service they offer, the provider-user enters their available schedule by entering day and time blocks using the available scheduling feature 48, followed by the provider-user entered schedule being automatically entered into the software platform's database 40. The provider's available schedule and service types are then displayed to the consumer in the master view list 42 and the find viewer 34. The provider-user is able to provide their profile photo, detail their work and education experience, and enter their work location in the provider profile feature 54. All provider-user features within the graphical user interface dashboard 28 of the software platform 10 are able to be selected by the provider-user from the provider dashboard 44. A new provider is added into the system by the provider-user completing the provider signup form feature located on the entrance screen of the software platform application 22 and 24. After the provider submits a provider sign-up form, the new provider's information on the form is reviewed for approval by the operator of the reservation software platform system. Once the operator approves the provider, a new profile is created for the provider, the provider's information is entered into the System, the provider is authenticated to accept bookings and fulfill bookings ordered by consumers using the software platform 10, and the provider is given access to the provider dashboard 44 within the graphical user interface dashboard 28 of the software platform application 10. The providers are individually limited to providing services for which they are licensed, educationally trained, and credentialed to provide, and each provider is verified by the system operator for having proper licensing documentation prior to be entered into the software platform system. The services that a provider may provide for instant booking in the reservation software platform system includes any type of birth or pregnancy related service, class or session, including but not limited to midwife services, at home birth services, birth center services, doula services, insemination services, lactation consulting services, birth coaching, breastfeeding sessions, childbirth classes, infant parenting classes, pregnancy exercise classes and sessions, pregnancy nutrition sessions, scheduled births to take place at birthing centers, scheduled home birth sessions, specialty birth sessions including water births, group and private breastfeeding classes, at home lactation consulting, at home insemination services, at home doula services, pregnancy classes, birthing classes, prenatal and postnatal classes.

I claim:

1. A reservation method as implemented on a software platform system for use by a consumer through a computer device, said method for booking and paying for a birth or pregnancy related class, session, or service with a birth-pregnancy provider listed within said software platform System with said birth-pregnancy provider being notified, dispatched and fulfilling the ordered services, class or session as booked by said consumer, said method comprising the steps of:
(1) displaying a list of available services and authorized birth-pregnancy providers available by location for selection by consumer using the software platform.
(2) prompting the consumer to select the type of birthing or pregnancy service, class, or session required by consumer.
(3) displaying available providers to said consumer based on the type of service selected by consumer.
(4) displaying the cost of selected service.
(5) prompting for said consumer to book and pay for the said consumer selected service fulfilled by said selected provider.
(6) prompting said consumer to select a payment option provided within the software platform to finalize the booking.
(7) initiating an electronic dispatch message to the said provider immediately after said consumer books the selected service.
(8) initiating an automated entry of the said consumer booked service into the said providers booking manager viewer within the software platform system.
(9) providing said provider with said consumer's provided detailed pregnancy due date information provided by said consumer, if applicable with said consumer.
(10) providing said consumer with said provider's direct emergency telephone number when a live birthing service is ordered including an at home birth service or a birth center service.

2. The method of claim 1, wherein said software platform may be on a computer device including a mobile device.

3. The method of claim 1, wherein said software platform may be accessed through a computer via the internet on a website with the said software platform application installed in the website.

4. The method of claim 1, wherein said software platform may be accessed via a mobile device having the said software platform application installed on the device.

5. The method of claim 1, wherein the providers are able to access the said software platform and enter their available schedule and provided services into said software platform system.

6. The method of claim 1, wherein the consumer and provider are able to create user profiles in the said software platform.

7. The method of claim 1, wherein the said providers include all types of birthing and pregnancy providers including midwives, doulas, birth center providers, lactation consultants, lactation counselors, birthing coaches, at home insemination providers, pregnancy nutrition providers, prenatal and postnatal care specialists, pregnancy class specialists, infant class providers, pregnancy fitness providers, birthing class providers, breastfeeding instructors, water birth specialists.

8. The method of claim 1, wherein the said provider is able to list and enter their availability schedule and type of class/session/service they provided into the software platform.

9. The method of claim 1, wherein the said consumer is able to find, select, schedule, reserve, book, and pay for the birth or pregnancy related class, session or service listed within the said software platform.

10. The method of claim 1, wherein the said provider will receive and fulfill consumer booked service assigned to said provider through the software platform after the said consumer selected, scheduled, and booked the class, session, or service.

11. The method of claim 1, wherein said provider will be charged a referral fee by the system operator for each booking assignment received from said software platform system.

12. The method of claim 1, wherein said provider will receive said payment for fulfilling said consumer booked service minus a referral fee charged by the system operator for each booking assignment received from said software platform system.

13. The method of claim 1, wherein said provider fulfillment of said consumer booked service will occur at either the said consumer own location or provider location as specified in said consumer service booked.
